(19) United States
(12) Patent Application Publication
(10) Pub. No.: US 2017/0340548 A1
(43) Pub. Date: Nov. 30, 2017

(54) STABLE DISPERSIONS COMPRISING DROPS OF A PERFUMING AGENT

(71) Applicant: CAPSUM, Marseille (FR)

(72) Inventors: Mathieu Goutayer, Saint Malo (FR); Javier Angel Tesan, Marseille (FR)

(73) Assignee: CAPSUM, Marseille (FR)

(21) Appl. No.: 15/536,403

(22) PCT Filed: Dec. 16, 2015
(86) PCT No.: PCT/EP2015/080010

§ 371 Date: Jun. 15, 2017

(30) Foreign Application Priority Data
Dec. 16, 2014 (FR) 1462525

(51) Int. Cl. A61K 8/81; A61K 8/04; A61K 8/11

(52) U.S. Cl. CPC A61K 8/8152; A61K 8/11; A61K 8/042; A61K 2800/52; A61K 2800/41

(57) ABSTRACT

The present invention relates to the use of at least one cross-linked polymer or a cross-linked copolymer, the polymer or copolymer comprising at least one unit derived from the polymerization of one of the monomers selected from the group consisting of acrylic acid, methacrylic acid, alkyl acrylate comprising from 1 to 30 carbon atoms and salts thereof, for stabilizing at least one perfuming agent in a composition comprising at least 3% by weight of perfuming agent(s), the perfuming agent being in the form of dispersed drops in an aqueous gel, the aqueous gel comprising water, a buffer having a pKa comprised from 4.0 to 9.0 and a base, and the size of the perfuming agent drops ranging from 0.2 μm to 5,000 μm.

The object of the present invention is stable dispersions comprising drops of perfuming agent(s), as well as their uses in the cosmetic field. The object of the present invention is more particularly perfuming compositions as stable dispersions comprising drops of perfuming agent(s), notably macroscopic and monodisperse, suspended in a continuous aqueous phase, said compositions being in particular without any polar solvent and/or surfactant.

The preparation of perfuming compositions is mainly based on the dissolution of aromatic compounds in a hydroalcoholic solution (mixture of water and alcohol, typically ethanol). The use of an alcohol such as ethanol gives the possibility, by its solvent properties, of solubilizing the aromatic compounds which are generally insoluble or sparsingly soluble in water.

However, alcohol has many drawbacks: it is a dermo-sensitizing agent, a drying agent, an irritating agent. Moreover, its use is not recommended for very young children. Furthermore, the regulations regarding the protection of the environment have recently become more strict concerning volatile compounds such as ethanol.

Perfuming compositions on an aqueous basis and without any ethanol are known. Such compositions are an alternative to the perfumes based on alcoholic solvents but to this day have not encountered the commercial success which was reckoned with. This notably explains the fact that these perfuming compositions on an aqueous basis and without any ethanol contain a strong level of surfactants, which is recognized as being an irritating factor and which may lead to a sensation of fat or tackiness on application and to constriction of the skin. Further, the surfactants may modify the olfactory profile of the perfuming agents.

Thus, to this day there exists a need in the field of perfumery and more generally of cosmetics for replacing the hydroalcoholic compositions and providing perfuming compositions without any alcohol and/or comprising a reduced or even zero amount of surfactants relatively to the present compositions.

To this day, there exists dispersions of droplets of an oily phase in an aqueous gel, notably as described in applications WO 2012/120043, FR 2 972 367 and FR 2 976 824. These dispersions are obtained by means of a microfluidic method.

In the case when the dispersed phase is complex like in the case when it mainly consists of perfume, a diffusion of the more hydrophilic molecules towards the continuous phase may occur. This phenomenon leads to a drop in the viscosity of the gel. A degradation will occur overtime, giving a non-suspensive finished product, potentially acid and with an unsuitable texture.

Therefore there exists a need for new dispersions comprising at least one perfuming agent and in particular without any polar solvent and/or surfactant which are stable, and this in spite of a high concentration of perfume(s).

The aim of the present invention is to provide a stable dispersion of drops comprising at least one perfuming agent dispersed in a continuous aqueous phase, in particular without any polar solvent and/or surfactant.

The aim of the present invention is also to provide a stable gel in the presence of perfume(s) having viscosities compatible with easy handling of the obtained product.

The aim of the present invention is also to provide a dispersion of drops of perfume(s) of a suitable viscosity, said dispersion may be formulated as a spray.

The aim of the present invention is also to provide a transparent dispersion of drops of perfume(s).

Thus, the present invention relates to the use of at least one cross-linked polymer or of at least one cross-linked copolymer comprising at least one unit derived from the polymerization of one of the monomers selected from the group consisting of acrylic acid, methacrylic acid, alkyl acrylate comprising from 1 to 30 carbon atoms and their salts, for stabilizing at least one perfuming agent in a dispersion comprising at least 3% by weight of perfuming agent(s), the perfuming agent(s) being in the form of drops dispersed in an aqueous gel, said aqueous gel comprising water, a buffer having a pKa comprised from 4.0 to 9.0 and a base, and the size of the drops of perfuming agents ranging from 0.2 μm to 5,000 μm.

According to an embodiment, within the scope of the use according to the invention, the aforementioned aqueous gel may also comprise a carbomer different from said cross-linked polymer or cross-linked copolymer.

The present invention also relates to a dispersion containing a dispersed phase comprising drops and a continuous aqueous phase, preferably as a gel, wherein:
the drops comprise at least one perfuming agent; and
the continuous aqueous phase comprises water, a buffer having a pKa comprised from 4.0 to 9.0, a base, preferably a mineral base, and at least one cross-linked polymer or cross-linked copolymer comprising at least one unit derived from the polymerization of one of the monomers selected from the group consisting of acrylic acid, methacrylic acid, alkyl acrylate comprising from 1 to 30 carbon atoms and salts thereof.

The present invention also relates to a dispersion containing a dispersed phase comprising drops and a continuous aqueous phase, preferably as a gel, wherein:
the drops comprise at least one perfuming agent and a shell, said shell comprising at least one carbomer and/or one cross-linked copolymer acrylates/C₁₀₋₃₀ alkyl acrylate; and
the continuous aqueous phase comprises water, a buffer having a pKa comprised from 4.0 to 9.0, a base, preferably a mineral base, and at least one cross-linked polymer or a cross-linked copolymer different from said carbomer and/or an aforementioned cross-linked copolymer acrylates/C₁₀₋₃₀ alkyl acrylate, said cross-linked polymer or cross-linked copolymer comprising at least one unit derived from the polymerization of one of the monomers selected from the group consisting of acrylic acid, methacrylic acid, alkyl acrylate comprising from 1 to 30 carbon atoms and salts thereof.

Surprisingly, it was observed that the use of at least one cross-linked polymer or cross-linked copolymer as mentioned above gives the possibility of stabilizing the perfuming agent(s) comprised in a dispersion as defined above, and this notably over a duration greater than one month and at temperatures comprised between 5° C. and 50° C., preferably between 10° C. and 60° C.

Within the scope of the present invention, the aforementioned dispersions may be equally designated by the term of “emulsions”.

According to the invention, the pH of the dispersion is typically greater than 6.5, and preferably comprised from 6.6 to 8.0, preferentially from 7.0 to 7.5.

A drop according to the invention consists of a core, also called the inside of the drop, if necessary surrounded by a shell which isolates the inside of the drop from the continuous phase of the dispersion.

According to an embodiment, the dispersions according to the invention do not comprise any surfactant. They are therefore differentiated, in this case, from the usual cosmetic dispersions/compositions.

Preferably, in the dispersions according to the invention, the size of the drops of perfuming agent(s) is comprised between 0.2 μm and 5,000 μm, and preferably between 20 μm and 2,500 μm, and preferentially between 800 μm and 1,500 μm.

According to an embodiment, the average diameter of the drops of the dispersed phase is comprised between 0.2 μm and 3,000 μm, preferably between 20 μm and 2,500 μm, and in particular between 800 μm and 1,500 μm.

According to an embodiment, a dispersion according to the invention is prepared by applying a “non-microfluidic” method, i.e. by simple emulsification. According to this embodiment, the size of the drops of the dispersed phase is less than 500 μm, or even less than 200 μm. Preferentially, the size of the drops is comprised between 0.5 μm and 50 μm, preferably between 1 μm and 20 μm.

According to this embodiment, the present invention gives the possibility of having drops with a reduced size, notably relatively to drops obtained by a microfluidic method. This small size of drops will have an effect on the texture. Indeed, a dispersion according to the invention, formed with finely dispersed drops has improved smoothness qualities.

According to another embodiment, a dispersion according to the invention is prepared by applying a “microfluidic” method, notably as described hereafter. According to this embodiment, the size of the drops of the dispersed phase is greater than 500 μm, or even greater than 1,000 μm. Preferentially, according to this embodiment, the size of the drops is comprised between 500 and 5,000, preferably between 700 and 3,000 μm, and better between 1,000 μm and 2,000 μm. As such, it was not obvious that dispersions comprising such drops with a size greater than 500 μm are stable.

Within the scope of the present invention, the term of “size” refers to the diameter, notably the average diameter of the drops.

Viscosity

The viscosity of the dispersions according to the invention may vary significantly which gives the possibility of obtaining various textures.

According to an embodiment, the dispersion according to the invention has a viscosity comprised from 1 mPa·s to 500,000 mPa·s, preferably from 10 to 300,000 mPa·s, and still more preferentially from 1,000 mPa·s to 100,000 mPa·s, as measured at 25° C. and at ambient pressure.

According to a preferred embodiment, the aforementioned dispersion has a viscosity comprised from 400 mPa·s to 20,000 mPa·s, preferably from 800 mPa·s to 15,000 mPa·s, as measured at 25° C. and at ambient pressure.

The viscosity is measured at room temperature, for example T=25° C.±2° C. and at ambient pressure, for example 1013 mbars, with the following method.

A viscosimeter of the Brookfield type, typically a digital viscosimeter Brookfield RVDV-E (torsional torque of the spring of 7187.0 dyne-cm), is used, which is a rotary viscosimeter with an imposed velocity provided with a mobile (designated as <<a Spindle>>). A velocity is imposed to the mobile in rotation and the measurement of the torque exerted on the mobile gives the possibility of determining the viscosity by knowing the geometry/shape properties of the mobile used.

For example a mobile of size No. 04 (Brookfield reference: RV4) is used. The corresponding shear rate to the measurement of the viscosity is defined by the mobile used and the speed of rotation of the latter.

The measurement of viscosity is carried out over 1 minute at room temperature (T=25° C.±2° C.). About 150 g of solution are placed in a beaker with a volume of 250 ml, having a diameter of about 7 cm so that the height of the volume occupied by the 150 g of solution is sufficient for reaching the marked gauge on the mobile. Next, the viscosimeter is started at a speed of 10 revolution/min and one waits until the displayed value on the screen is stable. This measurement gives the viscosity of the tested fluid, as mentioned within the scope of the present invention.

Dispersed Phase

As indicated above, the dispersions of the invention comprise a dispersed phase and a continuous aqueous phase, preferably as a gel.

According to the invention, the dispersed phase contains drops of at least one perfuming agent.

The term of “drop of perfuming agent(s)” refers to a drop comprising at least one perfuming agent. Preferably, according to the invention, such a drop comprises a shell.

A drop of the dispersed phase according to the invention typically comprises a core comprising at least one perfuming agent, surrounded by a shell, which isolates the inside of the drop (the core) from the aqueous phase of the dispersion. In particular, the shell represents the external layer of a drop.

According to an embodiment, said drops form the totality of the dispersed phase.

Perfuming Agent

As indicated above, the dispersions according to the invention comprise at least one perfuming agent (as drops).

According to the invention, the perfuming agent or perfume may be in the form of a mixture. Thus, the drops according to the invention may comprise a single perfuming agent (or single perfume) or a mixture of several perfuming agents (or mixture of several perfumes).

From among the perfuming agents, mention may notably be made of any type of perfume or fragrance, both of these terms being used here indifferently. These perfumes or fragrances are well known to one skilled in the art and notably include the aforementioned ones, for example, in S. Arctander, Perfume and Flavor Chemicals (Montclair, N.J., 1969), S. Arctander, Perfume and Flavor Materials of Natural Origin (Elizabeth, N.J., 1960) and in “Flavor and Fragrance Materials”, 1991 (Allured Publishing Co. Wheaton, Ill. USA). The perfumes used within the scope of the present invention may comprise natural products like extracts, essential oils, absolutes, resinoids, resins, concretes, etc. . . . as well as basic synthesis substances like hydrocarbons, alcohols, aldehydes, ketones, ethers, acids, esters, acetals, ketals, nitriles, etc . . . , including saturated and unsaturated compounds, aliphatic, alicyclic and heterocyclic compounds.

According to an embodiment, the perfuming agent comprises less than 25% by weight of linear or branched, saturated alcohol(s) or optionally comprising at least one unsaturation, based on the total weight of said perfuming agent. More particularly, according to an embodiment, the perfuming agent comprises less than 25% by weight of terpenic alcohol(s) based on the total weight of said perfuming agent.

According to an embodiment, the perfuming agent comprises less than 10%, or even less than 5%, by weight of aldehyde(s), based on the total weight of said perfuming agent.

According to an embodiment, the perfuming agent comprises less than 10%, or even less than 7.5%, by weight of compound(s) with a C log P of less than 2.1, based on the total weight of said perfuming agent.

According to an embodiment, the perfuming agent comprises at least 3%, or even at least 4%, by weight of linear and/or branched alcohol(s), and less than 25%, or even less than 20%, or further less than 15%, by weight of linear and/or branched alcohol(s), based on the total weight of said perfuming agent.

According to an embodiment, the perfuming agent comprises 4% by weight of linear and/or branched alcohol(s), based on the total weight of said perfuming agent.

According to an embodiment, the perfuming agent comprises 13% by weight of linear and/or branched alcohol(s) based on the total weight of said perfuming agent, relatively to the total weight of said perfuming agent.

According to an embodiment, the perfuming agent does not comprise any aldehyde.

According to an embodiment, the perfuming agent does not comprise any component with a C log P of less than 2.1.

According to an embodiment, a drop of perfuming agent(s) according to the invention comprises more than 60%, or even more than 70%, preferably more than 80%, and preferentially more than 90%, by weight of perfuming agent(s), said perfuming agent comprising:
less than 25% by weight of linear or branched alcohol(s), as defined above, based on the total weight of said perfuming agent(s);
less than 10%, or even less than 5%, by weight of aldehyde(s), based on the total weight of said perfuming agent(s); and
at least 10%, or even less than 7.5%, by weight of compound(s) with a C log P of less than 2.1, based on the total weight of said perfuming agent(s).

According to the invention, a drop of perfuming agent(s) according to the invention may comprise from 0.1% to 100%, preferably from 10% to 99.9% by weight of perfuming agent(s) based on the total weight of said drop.

A dispersion according to the invention may comprise from 0.1% to 20% by weight of perfuming agent(s), preferably from 1% to 15%, based on the total weight of the dispersion.

According to an embodiment, the content of perfuming agent(s) is of at least 3% by weight based on the total weight of the dispersion. In particular, the dispersion comprises from 5% to 10% by weight of perfuming agent(s) based on the total weight of said dispersion.

Solvent of a perfuming agent According to another embodiment, the dispersed phase may further comprise at least one solvent of perfuming agent(s), in particular a polar solvent, and better an alcoholic solvent.

Such a solvent may notably be selected from among propylene glycol, dipropylene glycol, diethyl phthalate, (tri)ethyl citrate, benzyl benzoate, benzyl acetate, benzylic alcohol, 2-(2-ethoxyethoxy)-1-ethanol, 1,2-alkanediols with 5 to 8 carbon atoms, butylene glycol, t-butylic alcohol, isopropyl palmitate, buteth-3, tributyl citrate, hexylene glycol, pentyl glycerol and mixtures thereof.

A dispersion according to the invention may comprise less than 5%, preferably from 0.01% to 4%, and better from 0.1% to 2%, by weight of solvent(s) of perfuming agent(s) based on the total weight of the dispersion.

According to another embodiment, the dispersed phase is without any solvent of perfuming agent(s) in particular without any polar solvent, and better any alcoholic solvent.

According to an embodiment, the dispersed phase may further comprise at least one oil or a solid fat at room temperature and ambient pressure, and mixtures thereof.

According to another embodiment, the dispersed phase is without any oil or without any solid fat at room temperature and at ambient pressure.

It should be noted that certain perfuming agent(s), in addition to their perfuming action, also act as a solvent of the cationic polymer as defined hereafter, notably amodimethicone.

Oil

According to the invention, the dispersed phase may further comprise at least one oil H1, notably wherein the cationic polymer defined hereafter is soluble.

By “oil” is meant a liquid fat at room temperature (25° C.).

As oils H1 which may be used in an emulsion according to the invention, mention may for example be made of:
hydrocarbon oils of animal origin, such as perhydrosqualene and squalane;
synthesis esters and ethers, notably from fatty acids, like the oils of formulae R1COOR2 and R1OR2 wherein R1 represents the radical of a C8-C29 fatty acid, and R2 represents a C3-C30 either branched or not, hydrocarbon chain, like for example Purcellin oil, isononyl isononanoate, isodecyl neopentanoate, isopropyl myristate, ethyl-2-hexyl palmitate, octyl-2-dodecyl stearate, octyl-2-dodecyl erucate, isostearyl isostearate; hydroxylated esters like isostearyl lactate, octylhydroxystearate, octyldodecyl hydroxystearate, diisostearyl-malate, triisocetyl citrate, heptanoates, octanoates, decanoates of fatty alcohols; polyol esters like propylene glycol dioctanoate, neopentylglycol diheptanoate and diethyleneglycol diisononanoate; and esters of pentaerythritol like pentaerythrityl tetraisostearate (Prisorine 3631);
linear or branched hydrocarbons, of mineral or synthetic origin, such as paraffinic oils either volatile or not, and derivatives thereof, vaseline, polydecenes, hydrogenated polyisobutene such as Parleam oil;
silicone oils, like for example polymethylsiloxanes (PDMS) either volatile or not with a liquid or pasty linear or cyclic silicone chain at room temperature, notably cyclopolydimethylsiloxanes (cyclomethicones) such as cyclohexasiloxane and cyclopentasiloxane; polydimethylsiloxanes (or dimethicones) including pendant alkyl, alkoxy or phenyl groups or with a silicone chain end, groups having from 2 to 24 carbon atoms; phenylated silicones like phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
fatty alcohols having from 8 to 26 carbon atoms, like cetyl alcohol, stearyl alcohol and mixtures thereof (cetylstearyl alcohol), or further octyldodecanol;
partly hydrocarbon and/or silicone fluorinated oils like those described in document JP-A-2-295912; and
mixtures thereof.

According to an embodiment, the oil H1 is selected from among esters of formula R1COOR2, wherein R1 represents the radical of a C8-C29 fatty acid, and R2 represents a C3-C30 hydrocarbon chain either branched or not.

According to an embodiment, the oil H1 is selected from among fatty alcohols having from 8 to 26 carbon atoms.

According to an embodiment, the oil H1 is selected from among the silicone oils, such as for example polydimethylsiloxanes (PDMS).

According to an embodiment, the oil H1 is selected from among hydrocarbon oils having from 8 to 16 carbon atoms, and notably C8-C16 branched alkanes (also called isoparaffins or isoalkanes), like isododecane (further called 2,2,4,4,6-pentamethylheptane), isodecane, isohexadecane, and for example, the oils sold under the commercial names of Isopars® or Permethyls®.

According to a preferred embodiment, the oil H1 is selected from the group consisting of isononyl isononanoate, dimethicone, isohexadecane, polydimethylsiloxane, octyldodecanol, isodecyl neopentanoate and mixtures thereof.

Preferably, the oil H1 is isononyl isononanoate.

According to an embodiment, the oil H1 is not a vegetable oil.

According to an embodiment, the oil H1 is not polydimethylsiloxane (PDMS), and preferably is not a silicone oil.

According to a preferred embodiment, a dispersion according to the invention comprises at least 1% by weight of H1 oil(s), preferably isononyl isononanoate, based on the total weight of said dispersion.

According to an embodiment, the H1 oil(s) content in the dispersed phase is comprised between 0% and 97%, preferably between 1% and 95%, better between 20% and 90%, and in particular between 50% and 80%, by weight based on the total weight of said dispersed phase.

According to an embodiment, the dispersed phase of the dispersions of the invention further comprise at least one hydrocarbon oil of vegetable origin H2. The fatty phase may comprise several H2 oils.

As vegetable oils H2, mention may notably be made of C4-C10 liquid triglycerides of fatty acids like triglycerides of heptanoic or octanoic acids or further, for example sunflower, maize, soya, pumpkin, grape pip, sesame, hazelnut, apricot, macadamia, arara, castor, avocado oils, triglycerides of caprylic/capric acids like those marketed by Stearineries Dubois and those available under the commercial names of “Miglyol 810”, “Miglyol 812” and “Miglyol 818” by Dynamit Nobel, jojoba oil, shea butter oil, and mixtures thereof.

Preferably, the oil H2 is selected from among oils rich in polyunsaturated fatty acids.

By “unsaturated fatty acids” in the sense of the present invention is meant a fatty acid comprising at least one double bond. More particularly these are fatty acids with long chains, i.e. which may have more than 14 carbon atoms. The unsaturated fatty acids may be in acid form, or in the form of a salt, such as for example their calcium salt, or further as derivatives, notably ester(s) of fatty acid(s).

Preferably, the oil H2 is selected from among oils rich in fatty acids with long chains, i.e which may have more than 14 carbon atoms, and better in unsaturated fatty acids including from 18 to 22 carbon atoms, in particular ω-3 and ω-6 fatty acids. Thus, advantageously, the vegetable oils are selected from among evening primrose, borage, blackcurrant pip, hemp, walnut, soybean, sunflower, wheat germs, fenugreek, rosehip, echium, argan, baobab, rice bran, sesame, almond, hazelnut, chia, seed oil flaxseed oil, olive oil, avocado oil, safflower, coriander, seed oil (notably Brassica naptus) oils, and mixtures thereof.

Preferably, the oil H2 is selected from among mat and not brilliant oils. Mention may be made as such of Moringa oil.

According to an embodiment, the content of H2 oil(s) in the dispersed phase is comprised between 0% and 40%, preferably between 0.1% and 25%, and in particular between 1% and 20%, by weight based on the total weight of said dispersed phase.

According to an embodiment, the mass ratio between the amount of H1 oil(s) and the ratio of H2 oil(s) ranges from 0.025 to 99.99, preferably from 0.8 to 90, and in particular from 2.5 to 80.

The dispersed phase may further comprise at least one other oil different from the H1 and H2 oils.

A dispersion according to the invention may comprise from 0% to 50%, preferably from 0.0001% to 50%, in particular from 0.1% to 40%, and better from 1% to 25%, by weight of oil(s) based on the total weight of said dispersion.

Solid fats According to an embodiment, the dispersed phase of a dispersion according to the invention comprises at least one solid fat at room temperature and at ambient pressure, notably selected from among waxes, slurry fatty acids, butters, and mixtures thereof.

Wax(es)

By “wax”, is meant in the sense of the invention, a lipophilic compound, solid at room temperature (25° C.), with a reversible change of state solid/liquid, having a melting point greater than equal to 30° C. which may range up to 120° C. The melting point may be measured according to the procedure described in FR 15 58849.

The waxes which may be used in a dispersion according to the invention are selected from solid, deformable or not waxes at room temperature, of animal, vegetable, mineral or synthetic origin and mixtures thereof.

It is notably possible to use hydrocarbon waxes like beeswax, lanolin wax, and China insect waxes; rice wax, Carnauba wax, Candellila wax, Ouricury wax, Alfa wax, cork fiber wax, sugarcane wax, Japan wax and sumac wax; montan wax, microcrystalline waxes, paraffins and ozokerite; polyethylene waxes, waxes obtained by Fisher-Tropsch synthesis and waxy copolymers as well as esters and mixtures thereof.

It is also possible to mention waxes obtained by catalytic hydrogenation of animal or vegetable oils having fatty, linear or branched C8-C32 chains.

Among the latter, may notably be mentioned hydrogenated jojoba oil, hydrogenated sunflower oil, hydrogenated castor oil, hydrogenated copper oil and hydrogenated lanolin oil, di-(trimethylol-1,1,1 propane) tetrastearate sold under the name of “HEST 2T-4S” by HETERENE, di-(trimethylol-1,1,1 propane) tetrabehenate sold under the name of HEST 2T-4B by HETERENE.

It is also possible to use waxes obtained by transesterification and hydrogenation of vegetable oils, such as castor or olive oil, like the waxes sold under the name of castor Phytowax 16L64® and 22L73® and Phytowax Olive 18L57 by SOPHIM. Such waxes are described in application FR-A-2792190.

It is also possible to use silicone waxes which may advantageously be substituted polysiloxanes, preferably with a low melting point.

From among the commercial silicone waxes of this type, mention may notably be made of those sold under the names of Abilwax 9800, 9801 or 9810 (GOLDSCHMIDT), KF910 and KF7002 (SHIN ETSU), or 176-1118-3 and 176-11481 (GENERAL ELECTRIC).

The silicone waxes which may be used may also be alkyl or alkoxydimethicones such as the following commercial products: Abilwax 2428, 2434 and 2440 (GOLDSCHMIDT), or VP 1622 and VP 1621 (WACKER), as well as (C20-C60) alkyldimethicones, in particular the (C30-C45) alkyldimethicones like the silicone oil sold under the name of SF-1642 by GE-Bayer Silicones.

It is also possible to use hydrocarbon waxes modified with silicone or fluorinated groups like for example: siliconyl candelilla, siliconyl beeswax and Fluorobeeswax from Koster Keunen.

The waxes may also be selected from fluorinated waxes.

Butter(s) or Pasty Fats

By “butter” (also called <<pasty fat>>) in the sense of the present invention is meant a lipophilic fat with a reversible change of state solid/liquid and including at the temperature of 25° C. a liquid fraction and a solid fraction, and at atmospheric pressure (760 mm Hg).

The pasty fat or butter may be selected from among the synthetic compounds and the compounds of vegetable origin. A pasty fat may be obtained by synthesis from starting products of vegetable origin.

The pasty fat is advantageously selected from among:
lanolin and its derivatives, such as lanolin alcohol, oxyethylenated lanolins, acetylated lanolin, lanolin esters such as isopropyl lanolate, oxypropylene lanolins,
the polymer or non-polymer silicone compounds such as polydimethysiloxanes of high molecular masses, polydimethysiloxanes with side chains of the alkyl or alkoxy type having from 8 to 24 carbon atoms, notably stearyl dimethicones,
polymer or non-polymer fluorinated compounds,
vinyl polymers, notably
olefin homopolymers,
olefin copolymers,
homopolymers and copolymers of hydrogenated dienes,
linear or branched oligomers, homo- or co-polymers of alkyl (meth)acrylates preferably having an alkyl C8-C30 group,
oligomers, homo and copolymers of vinyl esters having alkyl C8-C30 groups,
oligomers, homo and copolymers of vinylethers having C8-C30 alkyl groups,
fat-soluble polyethers resulting from the polyetherification between one or several C2-C100 diols, preferably C2-C50 diols,
esters and polyesters, and
mixtures thereof.

As butters of vegetable origin, mention may be made of those described in Ullmann's Encyclopedia of Industrial Chemistry (<<Fats and Fatty Oils>>, A. Thomas, published on 15 Jun. 2000, D01: 10.1002/14356007.a10_173, point 13.2.2.2. Shea Butter, Borneo Tallow, and Related Fats (Vegetable Butters)). More particularly mention may be made of C10-C18 triglycerides (INCI name: C10-18 Triglycerides) including at the temperature of 25° C. and at atmospheric pressure (760 mm Hg) a liquid fraction and a solid fraction, shea butter, shea butter Nilotica (Butyrospermum parkii), Galam butter, (Butyrospermum parkii), Borneo butter or fat or tengkawang tallow) (Shorea stenoptera), Shorea butter, Illipe butter, Madhuca or Bassia Madhuca longifolia butter, mowrah butter (Madhuca Latifolia), Katiau butter (Madhuca mottleyana), Phulwara butter (M. butyracea), mango butter (Mangifera indica), Murumuru butter (Astrocatyum murumuru), Kokum butter (Garcinia Indica), Ucuuba butter (Virola sebifera), Tucuma butter, Painya butter (Kpangnan) (Pentadesma butyracea), coffee butter (Coffea arabica), apricot butter (Prunus Armeniaca), Macadamia butter (Macadamia Temifolia), grain pip butter (Vitis vinifera), avocado butter (Persea gratissima), olive butter (Olea europaea), sweet almond butter (Prunus amygdalus dulcis), coco butter (Theobroma cacao) and sunflower butter, butter under the INCI name of Astrocaryum Murumuru Seed Butter, the butter under the INCI name of Theobroma Grandiflorum Seed Butter, and the butter under the INCI name of Irvingia Gabonensis Kernel Butter, jojoba esters (mixture of wax and of hydrogenated jojoba oil)(INCI name: Jojoba esters) and ethyl esters of shea butter (INCI name: Shea butter ethyl esters), and mixtures thereof.

Preferably, a dispersion according to the invention may comprise from 0% to 97% by weight, preferably from 0.5% to 70% by weight, in particular from 1% to 30% by weight, and better from 1% to 20% by weight, of solid fat(s) based on the total weight of the dispersed phase.

Shell of the Drops

As mentioned earlier, the drops according to the invention are preferably surrounded by a shell.

According to the invention, the obtained drops may have a very thin shell, notably with a thickness of less than 1% of the diameter of the drops.

The shell thickness is thus preferably less than 1 μm and is too small for being measured with optical methods.

According to an embodiment, the thickness of the shell of the drops is less than 1,000 nm, notably comprised from 1 to 500 nm, preferably less than 100 nm, advantageously less than 50 nm, preferentially less than 10 nm.

The measurement of the thickness of the shell of the drops of the invention may be carried out by small angle neutron scattering method (Small-Angle X-ray Scattering), as applied in Sato et al. J. Chem. Phys. 111, 1393-1401 (2007).

For this, the drops are produced by using deuterated water, and are then washed three times with a deuterated oil, such as for example a deuterated oil of the hydrocarbon type (octane, dodecane, hexadecane).

After washing, the drops are then transferred into the Neutron cell in order to determine the spectrum I(q); q being the wave vector.

From this spectrum, conventional analytic treatments are applied (REF) in order to determine the thickness of the hydrogenated (non-deuterated) shell.

According to an embodiment, the shell surrounding the drops of the dispersed phase is rigidified, which notably gives good resistance to the drops and reduces, or even prevents, their coalescence.

This shell is typically formed by coacervation, i.e. by precipitation of polymers charged with opposite charges. Within a coacervate, the bonds binding the charged polymers with each other are of the ionic type, and are generally stronger than bonds present within a membrane of the surfactant type.

The shell is formed by coacervation of at least two charged polymers of opposite polarity (or polyelectrolyte) and preferably in the presence of a first polymer, of the cationic type, and of a second polymer, different from the first polymer of the anionic type. Both of these polymers play the role of agents for rigidifying the membrane.

The formation of the coacervate between both of these polymers is generally caused by a modification of the conditions of the reaction medium (temperature, pH, concentration of reagents, etc.). The coacervation reaction results from the neutralization of both of these polymers charged with opposite polarities and allows the formation of a membrane structure by electrostatic interactions between the anionic polymer and the cationic polymer. The thereby formed membrane around each drop typically forms a shell which totally surrounds the core of the drop comprising the perfuming agent, and thereby isolates the core of the drop from the continuous aqueous phase.

Anionic Polymer

The continuous aqueous phase of a dispersion according to the invention may thus further comprise at least one anionic polymer, different from the cross-linked polymer or cross-linked copolymer comprising at least one unit derived from the polymerization of one of the following monomers: acrylic or methacrylic acid, alkyl methacrylate or acrylate comprising from 1 to 30 carbon atoms, or salts thereof and described in more detail hereafter.

Within the scope of the present description, by “polymer of the anionic type” is meant a polymer including chemical functions of an anionic type. This may also be referred to as an anionic polyelectrolyte.

By “chemical function of an anionic type”, is meant a chemical function AH capable of yielding a proton for obtaining a function A⁻. According to the conditions of the medium in which it is found, the polymer of the anionic type therefore includes chemical functions in the form of AH, or else in the form of its conjugate base A⁻.

As an example of chemical functions of the anionic type, mention may be made of carboxylic acid functions —COOH, optionally present in the form of a carboxylate anion —COO—.

As an example of a polymer of the anionic type, mention may be made of any polymer formed by the polymerization of monomers for which at least one portion bears chemical function of the anionic type, such as carboxylic acid functions. Such monomers are for example acrylic acid, maleic acid, or any ethylenically unsaturated monomer including at least one carboxylic acid function. These may for example be anionic polymers comprising monomer units including at least one chemical function of the carboxylic acid type.

Preferably, the anionic polymer is hydrophilic, i.e. soluble or dispersible in water. Within the scope of the invention, and unless indicated otherwise, by “hydrophilic” is meant the property according to which a given body is compatible with water or a polar solvent, i.e. it may accept water or said solvent, for forming with them a homogenous phase, for example a solution.

From among the examples of a polymer of the anionic type suitable for applying the invention, mention may be made of copolymers of acrylic acid or maleic acid and of other monomers, such as acrylamide, alkyl acrylates, C5-C8 alkyl acrylates, C10-C30 alkyl acrylates, C12-C22 alkyl methacrylates, methoxypolyethyleneglycol methacrylates, hydroxyester acrylates, crosspolymer acrylates.

According to an embodiment, the anionic polymer according to the invention is a carbomer or a cross-linked acrylates/C10-30 alkyl acrylate copolymer. Preferably, the anionic polymer according to the invention is a carbomer.

According to an embodiment, the shell of the drops comprises at least one anionic polymer, such as for example a carbomer.

Within the scope of the invention, and unless indicated otherwise, by “carbomer”, is meant an optionally cross-linked homopolymer, stemming from the polymerization of acrylic acid. This is therefore an optionally cross-linked poly(acrylic acid).

From among the carbomers of the invention, mention may be made of those marketed under the name of Tego® Carbomer 340FD from Evonik or Carbopol® 981 and carbopol Ultrez 10 from Lubrizol.

According to an embodiment, by “carbomer” or “carbomeric” or “Carbopol®” is meant a polymer of acrylic acid with a high cross-linked molecular weight with allyl sucrose or allyl ethers of pentaerythritol (handbook of Pharmaceutical Excipients, 5^{th} Edition, pill). For example, these are Carbopol® 910, Carbopol® 934, Carbopol® 934P, Carbopol® 940, Carbopol® 941, Carbopol® 71G, Carbopol® 980, Carbopol® 971P or Carbopol® 974P. According to an embodiment, the viscosity of said carbomer is comprised between 4,000 and 60,000 cP at 0.5% w/w.

The carbomers have other names: polyacrylic acids, carboxyvinyl polymers or carboxy polyethylene polymers.

According to the invention, each drop may comprise from 0.05% to 10% by weight of anionic polymer(s), notably carbomer(s), based on the total weight of said drop.

According to the invention, the anionic polymer may also be a cross-linked copolymer acrylates/C10-30 alkyl acrylate (INCI name: acrylates/C10-30 alkyl acrylate Crosspolymer) as defined above.

According to the invention, the emulsions according to the invention may comprise a carbomer and a cross-linked copolymer acrylates/C10-30 alkyl acrylate.

According to the invention, the aforementioned dispersion may further comprise from 0.01% to 10%, preferably from 0.1% to 2%, and preferentially from 0.15% to 0.5%, by weight of carbomer(s) and/or cross-linked copolymer(s) acrylates/C10-30 alkyl acrylate based on the total weight of said dispersion.

Cationic Polymer

According to an embodiment, the drops (the dispersed phase), and notably the shell of said drops, further comprise a polymer of the cationic type. They may also comprise several polymers of the cationic type. This cationic polymer is the one mentioned above which forms the shell by coacervation with the anionic polymer.

Within the scope of the present application, and unless indicated otherwise, is meant by “polymer of the cationic type”, a polymer including chemical functions of the cationic type. This may also be referred to as a cationic polyelectrolyte.

Preferably, the cationic polymer is lipophilic or fat-soluble.

Within the scope of the present application, and unless indicated otherwise, by “chemical function of the cationic type”, is meant a chemical function B capable of capturing a proton in order to obtain a BH⁺ function. Depending on the conditions of the medium in which it is found, the polymer of the cationic type therefore includes chemical functions in the form of B, or else in the form of BH⁺, its conjugate acid.

As an example of chemical functions of the cationic type, mention may be made of primary, secondary and tertiary amine functions, optionally present in the form of ammonium cations.

As an example of a polymer of the cationic type, mention may be made of any polymer formed by the polymerization of monomers for which at least one portion bears chemical functions of the cationic type, such as primary, secondary or tertiary amine functions.

Such monomers are for example aziridine, or any ethylenically unsaturated monomer including at least one primary, secondary or tertiary amine function.

From among the examples of polymers of the cationic type suitable for the application of the invention, mention may be made of amodimethicone, derived from a silicone polymer (polydimethylsiloxane, also called dimethicone), modified by primary amine and secondary amine functions, for example an amodimethicone having the following formula:

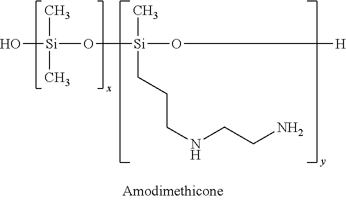

Mention may also be made of derivatives of amodimethicone, such as for example copolymers of amodimethicone, aminopropyl dimethicone, and more generally silicone polymers including amine functions.

Mention may be made of the copolymer of bis-isobutyl PEG-14/amodimethicone, Bis (C13-15 Alkoxy) PG-Amodimethicone, Bis-Cetearyl Amodimethicone and bis-hydroxy/methoxy amodimethicone.

Mention may also be made of polymers of the polysaccharide type comprising amine functions, such as chitosan or guar gum derivatives (guar hydroxypropyltrimonium chloride).

Mention may also be made of polymers of the polypeptide type comprising amine functions, such as polylysine.

Mention may also be made of polymers of the polyethyleneimine type comprising amine functions, such as linear or branched polyethyleneimine.

According to an embodiment, the drops, and notably the shell of said drops, comprise a cationic polymer which is a silicone polymer modified with a primary, secondary or tertiary amine function, such as amodimethicone.

According to an embodiment, the drops, and in particular the shell of said drops, comprise amodimethicone.

According to a particularly preferred embodiment, the cationic polymer fits the following formula:

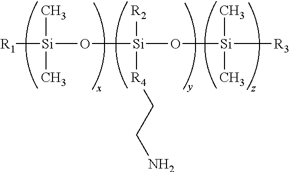

wherein:
R₁, R₂ and R₃, independently of each other represent OH or CH₃;
R₄ represents a group —CH₂— or a group —X—NH— wherein X is a divalent C3 or C4 alkylene radical;
x is an integer comprised between 10 and 5,000=, preferably between 30 and 1,000, and better between 80 and 300;
y is an integer comprised between 2 and 1,000, preferably between 4 and 100, and better between 5 and 20; and
z is an integer comprised between 0 and 10, preferably between 0 and 1, and better is equal to 1.

In the aforementioned formula, when R₄ represents a group —X—NH—, X is connected to the silicone atom.

In the aforementioned formula, R₁, R₂ and R₃ preferably represent CH₃.

In the aforementioned formula, R₄ is preferably a group —(CH₂)₃—NH—.

According to the invention, each drop may comprise from 0.01% to 10%, preferably from 0.05% to 10%, and better from 0.05% to 5%, by weight of cationic polymer(s), notably amodimethicone(s), based on the total weight of said drop (or dispersed phase).

According to the invention, the aforementioned dispersion may comprise from 0.05% to 5%, preferably from 0.1% to 2%, and preferentially from 0.15% to 0.5%, by weight of cationic polymer(s), notably amodimethicone(s), based on the total weight of said dispersion.

Continuous Aqueous Phase

As indicated earlier, the dispersions according to the invention comprise a continuous aqueous phase, preferably as a gel.

According to an embodiment, the aqueous phase has a viscosity comprised between 400 mPa·s and 20,000 mPa·s, preferably between 800 mPa·s and 15,000 mPa·s, as measured at 25° C.

This viscosity is measured according to the method described above.

The continuous phase of the dispersions comprise water.

In addition to distilled or deionized water, a water suitable for the invention may also be a natural source of water or a floral water.

According to an embodiment, the mass percentage of water of the continuous aqueous phase is of at least 40%, and at best at least 50%, notably comprised between 70% and 98%, preferentially comprised between 75% and 95% based on the total mass of said continuous phase.

Buffer

The continuous phase of the dispersions of the invention also comprises a buffer or a mixture of several buffers. According to the invention, the buffer used has a pKa comprised from 4.0 to 9.0.

Within the scope of the present invention, and unless indicated otherwise, is meant by “buffer” a chemical species which, in an aqueous solution, maintains the pH of the aqueous composition in which it is solubilized, and this in spite of the addition of small amounts of an acid or a base, or in spite of a dilution.

According to an embodiment, the buffer includes one or two sulfonic acid functions, preferably only one.

Preferably, the pKa of the buffer is comprised between 5.0 and 8.0, advantageously between 6.0 and 8.0.

According to an embodiment, the buffer is selected from the group consisting of buffers of phosphate, of 2-(N-morpholino)-ethane-sulfonic (MES) acid, of 2-amino-2-hydroxymethyl-1,3-propanediol, of 2-(bis(2-hydroxyethyl)amino) acetic acid, 4-(2-hydroxyethyl)-1-piperazine-ethane-sulfonic (HEPES) acid, sodium citrate and mixtures thereof.

Within the scope of the present invention, and unless specified otherwise, by “phosphate buffer” is meant a buffer comprising dihydrogenophosphate ions and hydrogenophosphate ions.

A phosphate buffer according to the invention may be prepared by dissolving monosodium or monopotassium phosphate and disodium or dipotassium phosphate in water.

As a phosphate buffer, mention may be made of PBS, which refers to the saline phosphate buffer (for “phosphate buffered saline”), prepared by dissolution of disodium phosphate (10 mM), monopotassium phosphate (1.76 mM), sodium chloride (137 mM) and potassium chloride (2.7 mM) in water. PBS has a pKa of 7.2 and gives the possibility of buffering an aqueous composition in a pH range ranging from 6.5 to 7.9.

As a phosphate buffer, it is also possible to mention the buffer prepared by dissolution of disodium phosphate (0.44% by mass) and of monopotassium phosphate (2.74% by mass) in water. Such a buffer has a pKa of 5.8.

In particular, the buffer is 4-(2-hydroxyethyl)-1-piperazine ethane sulfonic acid, notably called HEPES (CAS No. 7365-45-9). HEPES has a pKa of 7.5 and gives the possibility of buffering an aqueous composition in a pH range ranging from 6.8 to 8.2. 4-(2-hydroxyethyl)-1-piperazine ethane sulfonic acid is advantageous in that it stabilizes the dispersions according to the invention in the sought pH range and further contributes to preserving the integrity of the drops according to the invention.

According to an embodiment, the continuous aqueous phase of the dispersion comprises from 0.1% to 10%, preferably from 0.5% to 5%, by weight of buffer(s) based on the total weight of said continuous aqueous phase.

The dispersion according to the invention may comprise from 0.1% to 10% by weight of buffer(s), preferably from 0.5% to 5% by weight based on the total weight of the dispersion.

Base

The continuous aqueous phase of a dispersion according to the invention comprises at least one base. It may therefore comprise a single base or a mixture of several different bases. The presence of at least one base in said continuous aqueous phase notably contributes to adjusting the pH but also to increase the viscosity of the latter.

According to an embodiment, the base present in the aqueous phase is a mineral base.

According to an embodiment, the mineral base is selected from the group consisting of hydroxides of alkaline metals and hydroxides of earth-alkaline metals and mixtures thereof.

According to an embodiment, the base present in the aqueous phase is an organic base.

According to an embodiment, the organic base is selected from among the group consisting of ammonia, pyridine, triethanolamine, aminomethylpropanol, or further triethylamine, and mixtures thereof.

Preferably, the base is a mineral base, and better is a hydroxide of alkaline metals, and notably NaOH.

According to the invention, the dispersion of the invention may comprise from 0.01% to 10% by weight, preferably from 0.01% to 5% by weight, and preferentially from 0.05% to 1% by weight of base(s), preferably mineral base, and notably NaOH, based on the total weight of said dispersion.

Cross-Linked Copolymer/Polymer

The continuous aqueous phase according to the invention comprises at least one cross-linked polymer or at least one cross-linked copolymer, said cross-linked polymer or cross-linked copolymer comprising at least one unit derived from the polymerization of one of the following monomers: acrylic or methacrylic acid, alkyl acrylate or methacrylate comprising from 1 to 30 carbon atoms, or salts thereof, and being different from the anionic polymer described earlier when the latter is present.

The continuous phase may also comprise a mixture of cross-linked polymers or a mixture of cross-linked copolymers or further a mixture of cross-linked polymer(s) and of cross-linked copolymer(s).

According to the invention, the term of “unit derived from the polymerization of a monomer” means that the polymer or copolymer is a polymer or copolymer obtained by polymerization or copolymer of said monomer.

According to an embodiment, the cross-linked polymer or the cross-linked copolymer is a cross-linked polyacrylate.

The cross-linked copolymers and polymers of the invention are anionic.

According to an embodiment, the copolymer is a copolymer of unsaturated carboxylic acid and of unsaturated C₁-C₃₀ alkyl carboxylate, preferably C₁-C₄. Such a copolymer includes at least one hydrophilic unit of the olefinic unsaturated carboxylic acid type and at least one hydrophobic unit of the unsaturated carboxylic acid (C₁-C₃₀) alkyl ester type.

Preferably, these copolymers are selected from among those for which the hydrophilic unit of the olefinic unsaturated carboxylic acid type corresponds to the monomer of the following formula (I):

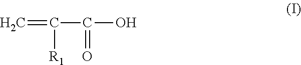

wherein: R₁ designates H or CH₃ or C₂H₅, i.e. units of acrylic acid, methacrylic acid or ethacrylic acid,
and for which the hydrophobic unit of the unsaturated carboxylic acid (C₁-C₃₀) alkyl ester type corresponds to the monomer of the following formula (II):

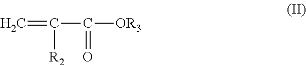

wherein: R₂ designates H or CH₃ or C₂H₅ (i.e. acrylate, methacrylate or ethacrylate units) and preferably H (acrylate units) or CH₃ (methacrylate units), R₃ designating a C₁-C₃₀ alkyl radical, and preferably C₁-C₄ alkyl radical, or a group —[CH₂—CH₂—O]ₙ—(CH₂)ₘ—CH₃ with:
n is an integer comprised between 1 and 100, preferably between 5 and 70, better between 10 and 50, and more particularly between 20 and 30; and
m is an integer, either identical or different from n, comprised between 1 and 30, preferably between 5 and 25, and better between 15 and 23.

Among this type of copolymers, those formed from a mixture of monomers will more particularly be used, comprising:
(i) essentially acrylic acid,
(ii) an ester of formula (II) described above and wherein R₂ designates H or CH₃, R₃ designating an alkyl radical having from 1 to 4 carbon atoms,
(iii) and a cross-linking agent, which is a well known copolymerizable polyethylene unsaturated monomer, such as diallyl phthalate, trimethylolpropane tri(meth)acrylate, diallyl itaconate, diallyl fumarate, diallyl maleate, zinc (meth)acrylate, allyl (meth)acrylate, divinylbenzene, (poly)ethyleneglycol dimethacrylate, methylene-bis-acrylamide, and castor oil.

According to an embodiment, the polymer or the copolymer is a polymer or copolymer of acrylic acid and/or methacrylic acid, and/or of alkyl acrylate comprising from 1 to 30 carbon atoms, preferably from 1 to 4 carbon atoms, and/or of alkyl methacrylate comprising from 1 to 30 carbon atoms, preferably from 1 to 4 carbon atoms.

According to an embodiment, the cross-linked copolymer is a cross-linked copolymer of methacrylic acid and of alkyl acrylate comprising from 1 to 4 carbon atoms, preferably 2 carbon atoms.

Within the scope of the invention, and unless indicated otherwise, by <<cross-linked copolymer of methacrylic acid and of alkyl acrylate comprising from 1 to 4 carbon atoms>>, is meant a cross-linked copolymer resulting from the polymerization of a monomer of methacrylic acid and of a monomer of alkyl acrylate comprising from 1 to 4 carbon atoms.

Preferably, in this copolymer, the methacrylic acid represents from 20% to 80% by weight, preferably from 35% to 65% by weight of the total weight of the copolymer.

Preferably, in this copolymer, the alkyl acrylate represents from 15% to 80% by weight, preferably from 35% to 65% by weight of the total weight of the copolymer.

In particular, the alkyl acrylate is selected from among alkyl methacrylate, ethyl acrylate and butyl acrylate.

According to an embodiment, the cross-linked polymer or the cross-linked copolymer according to the invention, present in the continuous aqueous phase, is selected from the group consisting of the following polymers or copolymers: Acrylate Copolymer, Acrylate crosspolymer-4, Acrylate crosspolymer-3, Polyacrylate-2 Crosspolymer and Polyacrylate-14 (INCI names).

Among said polymers above, are most particularly preferred according to the present invention, the products sold by LUBRIZOL under the commercial names Fixate Superhold (INCI name=Polyacrylate-2 Crosspolymer), Fixate Freestyle Polymer (INCI name=Acrylates crosspolymer-3), Carbopol® Aqua SF1 (INCI name=Acrylates copolymer) and Carbopol® Aqua SF2 (INCI name=Acrylates crosspolymer-4), or further the one sold by Croda Inc. under the commercial name of Volarest™ FL.

Preferably, the cross-linked polymer or the cross-linked copolymer is selected from among Carbopol® Aqua SF1 (INCI name=Acrylates copolymer) and Carbopol® Aqua SF2 (INCI name=Acrylates crosspolymer-4).

In particular, this is Carbopol® Aqua SF1 (INCI name=Acrylates copolymer).

In particular, this is Carbopol® Aqua SF2 (INCI name=Acrylates crosspolymer-4).

According to an embodiment, the cross-linked copolymer is selected from among the cross-linked copolymers of acrylic or methacrylic acid and of alkyl acrylates comprising from 1 to 4 carbon atoms.

According to the invention, the dispersion of the invention may comprise from 0.1% to 10% by weight, preferably from 0.5% to 8% by weight, and preferentially from 1% to 3% by weight of cross-linked polymer(s) or cross-linked copolymer(s) based on the total weight of said dispersion.

Additional Compound(s)

The dispersions of the invention may further comprise powders, flakes, coloring agents, preservatives, humectants, stabilizers, chelators, emollients etc. . . . or any usual cosmetic additive, and mixtures thereof.

The dispersions according to the invention may further comprise at least one active ingredient, preferably selected from among hydrating agents, healing agents, depigmenting agents, UV filters, desquamating agents, antioxidant agents, active ingredient stimulating synthesis of the dermal and/or epidermal macromolecular agents, dermodecontracting agents, anti-sweating agents, anti-ageing agents, and mixtures thereof.

Of course, one skilled in the art will make sure to select the additional compound(s) and/or their amount depending on the aqueous or fat nature of the relevant phase and/or in such a way that (i) the advantageous properties of a dispersion according to the invention and (ii) the integrity of the drops forming said dispersion are not or substantially not altered by the contemplated adjunction. These adjustments fall under the skills of one skilled in the art.

In particular, according to the invention, the drops, and notably the core of the drops, may further comprise at least one compound selected from among: the coloring agents, the stabilizers, the chelators, the preservatives, the emollients, the modifying agents selected from among texture, viscosity, pH, osmotic force agents or refractive index modifications, and mixtures thereof.

From among the preservatives, mention may notably be made of phenoxyethanol, pentylene glycol and EDTA.

According to an embodiment, the dispersions according to the invention comprise at least one preservative, and preferably a mixture of several preservatives.

Preferably, the weight content of preservative(s) is comprised between 0.1% and 10%, preferably between 0.5% and 5%, based on the total weight of said dispersion.

According to an embodiment, the dispersions of the invention further comprise glycerol. Preferably, the dispersions of the invention comprise at least 5% by weight of glycerol based on the total weight of said dispersions. Indeed, beyond this texture, the dispersions according to the invention provide another advantage relatively to <<conventional>> emulsions since they allow the use of glycerol, which furthermore is in high contents.

In particular they may comprise glycerol in a content greater than or equal to 10%, greater than or equal to 20%, greater than or equal to 30%, greater than or equal to 40%, or even up to 50%, by weight, based on the total weight of said dispersion.

For obvious reasons, glycerol is present at the aqueous phase of an emulsion according to the invention.

A dispersion according to this embodiment is of particular interest since it gives a perfuming composition as such additional exacerbated hydration properties.

Preparation Methods

The dispersions according to the invention may be prepared by different methods.

Thus, the dispersions according to the invention have the advantage of being able to be prepared according to a simple <<non-microfluidic>> method, i.e. by simple emulsification.

Like in a conventional emulsion, an aqueous solution and a fat solution are prepared separately. This is the addition with stirring of the fat phase into the aqueous phase which generates the direct emulsion. In the case of the present invention, the solution/fatty phase is featured by a solution/phase comprising at least one perfuming agent.

The dispersions according to the invention may also be prepared according to a microfluidic method, notably as described in the international applications WO 2012/120043 or WO 2015/055748.

According to this embodiment, the drops have a uniform size distribution. Preferably, the dispersions of the invention consist of a population of monodispersed perfuming agent drops, notably such that they have an average diameter D̅ comprised from 20 μm to 2 500 μm, in particular from 500 μm to 5,000 μm, and a variation coefficient Cv of less than 10%, or even less than 3%.

Within the scope of the present description, by “monodispersed drops” are meant the fact that the population of drops of the dispersion according to the invention have a uniform size distribution. Monodispersed drops have good monodispersity. Conversely, drops having poor monodispersity are said to be “polydispersed”.

According to an embodiment, the average diameter D̅ of the drops is for example measured by analyzing a photograph of a batch consisting of N drops, with an image processing piece of software (Image J). Typically, according to this method, the diameter is measured in pixels, and then reduced to μm, depending on the dimension of the container containing the drops of the dispersion.

Preferably, the value of N is selected to be greater than or equal to 30, so that this analysis reflects in a statistically significant way the distribution of the diameters of the drops of said emulsion.

The diameter Di of each drop is measured, and then the average diameter D̅ is obtained by calculating the arithmetic mean of these values:

$$\overline{D} = \frac{1}{N} {\sum }_{i = 1}^{N} {D}_{i}$$

From these values Dᵢ, it is also possible to obtain the standard-deviation a of the diameters of the drops of the dispersion:

$$σ = \sqrt{\frac{{\sum }_{i = 1}^{N} {( {D}_{i} - \overline{D} )}^{2}}{N}}$$

The standard-deviation 6 of a dispersion reflects the distribution of the diameters Dᵢ of the drops of the dispersion around the average diameter D̅.

By being aware of the average diameter D̅ and of the standard-deviation 6 of a dispersion, it is possible to determine that 95.4% of the population of drops are found in the interval of diameters [D̅−2σ;D̅+2σ] and that 68.2% of the population is found in the interval [D̅−σ;D̅+σ].

In order to characterize the monodispersity of the dispersion according to this embodiment, it is possible to calculate the variation coefficient:

$${C}_{v} = \frac{σ}{\overline{D}}$$

This parameter reflects the distribution of the diameters of the drops versus the average diameter of the latter.

The variation coefficient Cᵥ of the diameters of the perfuming agent drops according to this embodiment is less than 10%, preferably less than 5%, or even less than 3%.

Alternatively, the monodispersity may be shown by placing a dispersion sample in a flask with constant circular section. Mild stirring by rotation by a quarter of a turn over half a second around the axis of symmetry crossing the flask, followed by resting for half a second is carried out, before repeating the operation in the reverse direction, and this four times in succession.

The drops of perfuming agent(s) are organized in a crystalline form when they are monodispersed. Thus, they have a stack along a unit which is repeated along the three dimensions. It is then possible to observe, a regular stack which indicates good monodispersity, an irregular stack expressing the polydispersity of the dispersion.

The presence, in the dispersed phase, of solid fat(s) at room temperature and ambient pressure, as contemplated previously, may require adjustments at the method for preparing a dispersion according to the invention. In particular, the method for preparing such a dispersion according to the invention may comprise a heating step (between 40° C. and 150° C., notably between 50° C. and 90° C.) of the dispersed phase before mixing/contacting said dispersed phase with the aqueous phase and, if necessary, and in the case of a <<non-microfluidic>> method as mentioned above, the maintaining of this heating during stirring until the sought dispersion is obtained.

These adjustments fall under the general skills of one skilled in the art.

In the case of a dispersion according to the invention, the solutions (or fluids) used for making up the continuous aqueous phase and the dispersed phase are respectively designated as External Fluid (FE) and Internal Fluid (FI):
the fluid FI comprises at least one perfuming agent and further optionally, at least one first polymer precursor of the coacervate, notably a cationic polymer, and in particular an amodimethicone, at least one oil, at least one solid fat at room temperature and ambient pressure and/or at least one additional compound, notably as defined earlier;
the fluid FE comprises at least water and further optionally at least one cross-linked polymer or cross-linked copolymer comprising at least one unit derived from the polymerization of one of the monomers selected from the group consisting of acrylic acid, methacrylic acid, alkyl acrylate comprising from 1 to 30 carbon atoms and salts thereof, at least a second polymer precursor of the coacervate, different from the cross-linked polymer or a cross-linked copolymer mentioned above and different from the first polymer precursor of the coacervate, notably an anionic polymer, and in particular a carbomer, at least one aforementioned additional compound, or even preservatives and/or other soluble products in water such as glycerol;
the buffer(s) having a pKa comprised from 4.0 to 9.0, the base, preferably a mineral base, and the cross-linked polymer(s) or cross-linked copolymer(s) comprising at least one unit derived from the polymerization of one of the monomers selected from the group consisting of acrylic acid, methacrylic acid, alkyl acrylate comprising from 1 to 30 carbon atoms and salts thereof (notably if the fluid FE above is without any of them) are injected into the aqueous external fluid FE after formation of the dispersion according to the invention, and therefore after formation of the drops, and therefore after mixing the fluids FI and FE.

According to an embodiment, the method for preparing a dispersion according to the invention comprises a step for forming drops comprising:
the putting into contact of a fluid FE and of a fluid FI as defined above;
the formation of the drops of dispersed phase, consisting of the fluid FI, dispersed in a continuous aqueous phase consisting of fluid FE, said drops comprising a shell insulating the core of the drops of the dispersed phase of the dispersion; and
successive or simultaneous addition of at least one cross-linked polymer or cross-linked copolymer comprising at least one unit derived from the polymerization of one of the monomers selected from the group consisting of acrylic acid, methacrylic acid, alkyl acrylate comprising from 1 to 30 carbon atoms and salts thereof (notably if the fluid FE is without any of them), of at least one buffer having a pKa comprised from 4.0 to 9.0 and of at least one base, preferably a mineral base.

According to an embodiment wherein the dispersion is prepared according to a microfluidic method and wherein the fluid FI comprises at least the first polymer precursor of the aforementioned coacervate, the step for putting into contact the fluid FE and the fluid FI as defined above may further comprise the presence of an intermediate fluid miscible with the fluid FI, as described in WO 2012/120043. This intermediate fluid is intended to form a film around the drop formed by the fluid FI in the fluid FE. Thus, the intermediate fluid delays the diffusion of the first polymer precursor of the coacervate until the intermediate fluid is mixed with the fluid FI and thus ensures the formation of very stable drops stabilized by a very thin shell without obstruction of the microfluidic device.

According to an embodiment, the intermediate fluid may comprise at least one perfuming agent, identical with or different from the one present in the fluid FI.

According to an embodiment, when the FI comprises at least one solid fat at room temperature and ambient pressures as described earlier, the method for preparing a dispersion according to the invention may further comprise a step for heating the fluid FI, to a temperature comprised from 40° C. to 150° C., preferably from 50° C. to 90° C., prior to the aforementioned step for forming the drops, and therefore before mixing/contacting said fatty phase with the continuous aqueous phase.

This embodiment, in the case of a “non-microfluidic” method as mentioned above, may require the maintaining of this heating step after mixing/putting said fatty phase in contact with the continuous aqueous phase during stirring until the sought emulsion is obtained.

This embodiment, in the case when the emulsion is prepared according to a microfluidic method, is advantageous, notably in that it gives the possibility of doing without the presence of the intermediate fluid described above.

According to this embodiment, the preparation method may further comprise, between the heating step and the step for forming drops, a step consisting of lowering the temperature of the fluid FI, if necessary down to room temperature.

Uses of the Dispersion

Preferably, a dispersion according to the invention may be used directly, at the end of the aforementioned preparation methods, as a notably cosmetic composition. The dispersion according to the invention, when it is prepared by means of a micro-fluidic method as described above, may also be used as a composition, notably a cosmetic composition, after separation of the drops and re-dispersion of the latter into a second suitable phase.

The invention also relates to the use of a dispersion according to the invention for preparing a composition, notably a cosmetic composition.

Thus, the present invention also relates to a cosmetic composition comprising an aforementioned dispersion, associated with a physiologically acceptable medium.

Within the scope of the invention, and unless indicated otherwise, by “physiologically acceptable medium”, is meant a medium suitable for cosmetic applications, and notably suitable for applying a composition of the invention on the skin and/or on the hair. The physiologically acceptable medium is generally adapted to the nature of the support on which the composition should be applied, as well as to the aspect under which the composition has to be conditioned.

The cosmetic compositions according to the invention may be care products, solar protective products, cleaning products (makeup removal), hygiene or skin makeup products.

These compositions are therefore intended to be notably applied on the skin.

Thus, the present invention also relates to the non-therapeutic cosmetic use of an aforementioned cosmetic dispersion or composition, as a makeup, hygiene, cleansing product, care and/or perfuming of keratinous materials, notably of the skin.

According to an embodiment, the dispersions or compositions of the invention are in the form of a foundation, of a makeup-removal agent, of a face and/or skin and/or hair care product, of an anti-ageing care product, of a solar protective product, of a fatty skin care product, of a whitening care product, of a hydrating care product, of a BB cream, of a tinted cream or foundation, a face cleanser and/or body care, a shower gel or a shampoo.

A care composition de soin according to the invention may be in particular a solar composition, a care cream, a cream/perfuming composition, a serum or a deodorant.

The dispersions or compositions according to the invention may be in diverse forms, notably as a cream, a balm, a lotion, a serum, a gel, a gel-cream or further a mist.

Upon application on a keratinous material, in particular the skin, of a dispersion or a composition according to the invention, the drops of the dispersion destabilize very rapidly, typically under the shearing generated by the fingers on the keratinous material, without opposing any resistance. This behavior upon application has the advantage of contrasting with the solid and granular visual aspect of the drops of the dispersion.

According to a particular embodiment, a dispersion according to the invention is not dedicated to hair care. In other words, a dispersion according to the invention is not a capillary composition, a shampoo or a hair conditioner.

The present invention also relates to a non-therapeutic method for cosmetic processing of a keratinous material, in particular the skin, comprising a step for application on the keratinous material, in particular the skin, of at least one layer of a dispersion or of at least one aforementioned cosmetic composition.

In the whole application, the wording “comprising one” or “including one” means “comprising at least one” or “including at least one” unless specified otherwise.

In all the description above, unless indicated otherwise, the term <<comprised between x and y>> corresponds to an inclusive range, i.e. the values x and y are included in the range.

The following examples give the possibility of illustrating the invention without however limiting it.

### EXAMPLES

Raw Materials

| Name | INCI name | Provider |
|---|---|---|
| Carbomer 340FD | Carbomer (carbomer) | Evonik |
| Carbopol ® 981 | Carbomer (carbomer) | Lubrizol |
| Fixate Superhold | Polyacrylate-2 Crosspolymer | Lubrizol |
| Fixate Freestyle | Acrylate crosspolymer-3 | Lubrizol |
| Polymer | | |
| Carbopol ® Aqua SF1 | Acrylate copolymer | Lubrizol |
| Carbopol ® Aqua SF2 | Acrylate crosspolymer-4 | Lubrizol |
| KF8004 | Amodimethicone | Shin-Etsu |
| Soda | Sodium hydroxide | Panreac |
| Microcare ® PE | Phenoxyethanol | Thor |
| Microcare ® Emollient | Pentylene glycol | Thor |
| PTG | | |
| Edeta ® BD | Disodium EDTA | BASF |
| Hepes-Luv | Hydroxyethylpiperazine Ethane | Hopax |
| | Sulfonic Acid Hydro (HEPES) | |
| Perfume | Fragrance | Givaudan |
| DUB ININ | Isononyl Isononanoate | Stéarinerie |
| | | Dubois |
| Water | Aqua | — |

The perfume used comprises 4% by weight of linear and/or branched alcohols linéaires and 96% by weight of other perfume ingredients (and therefore 0% by weight of aldehydes and 0% by weight of ingredients with C log P<2.1).

### Example 1: Preparation of a Perfume Dispersion without Coacervation

This example consisted in preparing a gel suspending perfume droplets without stabilization of the latter by a coacervate membrane.

The composition of Example 1 consists of the following ingredients (cf. table below):

| Name | INCI name | % w/w |
|---|---|---|
| Osmosed water | Aqua | 83.68% |
| Perfume | Fragrance | 10% |
| Crosspolymer | Acrylate crosspolymer-4 | 2.70% |
| Carbopol Aqua SF2 | | |
| Microcare ® PE | Phenoxyethanol | 0.72% |
| Microcare ® emollient | Pentylene glycol | 1.80% |
| PTG | | |
| Hepes | Hydroxyethylpiperazine | 0.90% |
| | Ethane Sulfonic Acid Hydro | |
| Edeta ® BD | Disodium EDTA | 0.01% |
| NaOH | Sodium Hydroxide | 0.19% |

| | |
|---|---|
| Total | 100.00% |

The composition of Example 1 is prepared according to the following procedure:

The final half-water mixture is mixed with the preservatives. Stirring is performed by means of a de-flocculating type blade for 15 min at 300 rpm.

The Crosspolymer is added while maintaining mild stirring.

A solution is prepared separately with Hepes, the soda and the remainder of water which will be added slowly to the previous solution still with stirring.

Finally, the perfume is slowly incorporated and stirring is continued until total dispersion.

| | Time (in days) | | | | |
|---|---|---|---|---|---|
| | 0 | 7 | 14 | 21 | 28 |
| pH | 7.17 | 7.1 | 6.78 | 6.74 | 6.45 |
| Viscosity | 1,576 | 1,620 | 1,712 | 1,660 | 1,308 |
| (mPa · s) | | | | | |

The viscosity is measured according to the method indicated above in the description.

This composition proves to be advantageously stable for at least one month at 50° C. Even if the pH was lowered, the viscosity of the gel remained stable over time and at 50° C.

This example therefore gives the possibility of showing that the use of a cross-linked copolymer or of a cross-linked polymer according to the invention advantageously gives the possibility of stabilizing the compositions comprising a high amount of perfume.

### Example 2: Preparation of a Perfuming Composition

The perfuming composition of Example 2 (perfume serum) is a pumpable perfumed product which is stable over time.

The composition of Example 2 consists of the following ingredients (cf. table below):

| | | | % by weight |
|---|---|---|---|
| | | | based on the |
| | | | total weight of |
| Name | INCI name | | the composition |
| Osmosed water | Aqua | | 83.72% |
| Phase A | Isononyl | 10.45% | 1.05% |
| | Isononanoate | | |
| | Perfume | 89.10% | 8.91% |
| | Amodimethicone | 0.45% | 0.05% |
| | Total | 100.00% | |

| | | |
|---|---|---|
| Carbomer | Carbomer | 0.18% |
| Carbopol 981 from | | |
| Lubrizol | | |
| Crosspolymer | Acrylate crosspolymer-4 | 1.80% |
| Microcare ® PE | Phenoxyethanol | 0.90% |
| Microcare ® emollient | Pentylene glycol | 2.25% |
| PTG | | |
| Hepes | Hydroxyethylpiperazine | 0.90% |
| | Ethane Sulfonic Acid Hydro | |
| Edeta ® BD | Disodium EDTA | 0.01% |
| NaOH | Sodium Hydroxide | 0.23% |

| | |
|---|---|
| Total | 100.00% |

The composition of Example 2 is prepared according to the following procedure:

Half of the water and the preservatives are mixed. Stirring is performed by means of a de-flocculating type blade for 15 min at 300 rpm.

The stirring is stopped for incorporating the carbomer, one waits for 1h for the hydration of the latter in order to next stir by means of a de-flocculator rapidly but without incorporating any air bubbles for 2h.

The crosspolymer is added and is stirred until complete dispersion.

A solution with the Hepes, the soda and the remainder of water which has just been slowly added to the preceding solution always with stirring is prepared separately.

Finally, the phase A is prepared by mixing isononyl isononanoate, amodimethicone and perfume. This phase A is then incorporated to the aqueous phase with moderate stirring.

Alternatively, the solution with Hepes, the soda and the remainder of water may be added after dispersion of the phase A into the carbomer solution.

The final composition of the perfuming composition of Example 2 is the following:

| | | Flow rate | |
|---|---|---|---|
| Fluid | Composition | (g/min) | % w/w |
| IF | 99.50% Perfume | 3.97 | 9.48% |
| | 0.5% Amodimethicone | | |
| MF | 100% DUB ININ | 0.4 | 0.96% |
| OF | 0.25% Carbomer | 30 | 71.65% |
| | 1% Microcare ® PE | | |
| | 2.5% Microcare ® PTG | | |
| | 0.01% Edeta ® | | |
| | QSP 100% deionized water | | |
| Crosspolymer | 13.39% Acrylates crosspolymer-4 | 5.6 | 13.37% |
| | 0.019% NaOH | | |
| | QSP 100% deionized water | | |
| Base | 4.81% NaOH | 1.9 | 4.54% |
| | 19.77% Hepes | | |
| | QSP 10% deionized water | | |

The stability of this composition was studied at 50° C. for several days. The results in terms of pH and of viscosity are provided in the following table:

| | Time (in days) | | | | |
|---|---|---|---|---|---|
| | 0 | 7 | 14 | 21 | 27 |
| pH | 7.4 | 7.18 | 6.92 | 6.76 | 6.67 |
| Viscosity | 2,450 | 2,580 | 2,760 | 2,470 | 2,360 |
| (mPa · s) | | | | | |

The viscosity is measured according to the method described in Example 1.

This composition prove to be advantageously stable for at least one month at 50° C. Indeed, the viscosity and the pH did not vary much over time and at 50° C. Consequently, the gel is not very acidified and has retained its suspensive properties.

Furthermore, the composition according to Example 2 has an olfactory profile similar to the one of a conventional perfuming composition, i.e. hydroalcoholic and comprising an identical perfume content.

### Example 3: Preparation of a Perfuming Composition

The perfuming composition of Example 3 (perfume serum) is a perfumed product (which may be powdered) which is stable over time.

The composition of Example 3 consist of the following ingredients (cf. table below):

| | | | % by weight |
|---|---|---|---|
| | | | based on the |
| | | | total weight of |
| Name | INCI name | | the composition |
| Osmosed water | Aqua | | 86.21% |
| Phase A | Isononyl | 16.12% | 1.47% |
| | Isononanoate | | |
| | Perfume | 83.41% | 7.28% |
| | Amodimethicone | 0.46% | 0.04% |
| | Total | 100.00% | |

| | | |
|---|---|---|
| Carbomer | Carbomer | 0.14% |
| Crosspolymer | Acrylates crosspolymer-4 | 1.38% |
| Microcare ® PE | Phenoxyethanol | 0.74% |
| Microcare ® emollient | Pentylene glycol | 1.84% |
| PTG | | |
| Hepes | Hydroxyethylpiperazine | 0.7% |
| | Ethane Sulfonic Acid Hydro | |
| Edeta ® BD | Disodium EDTA | 0.01% |
| NaOH | Sodium Hydroxide | 0.14% |

| | |
|---|---|
| Total | 100.00% |

The composition of Example 3 is prepared according to the procedure of Example 2.

The final composition of the perfuming composition of Example 3 is the following:

| | | Flow rate | |
|---|---|---|---|
| Fluid | Composition | (g/min) | % w/w |
| IF | 90% Perfume | 14.82 | 7.96% |
| | 0.5% Amodimethicone | | |
| | 9.5% DUB ININ | | |
| MF | 100% DUB ININ | 1.28 | 0.7% |
| OF | 0.19% Carbomer | 135 | 72.05% |
| | 1% Microcare ® PE | | |
| | 2.5% Microcare ® PTG | | |
| | 0.01% Edeta ® | | |
| | 0.005% NaOH | | |
| | QSP 100% deionized water | | |
| Crosspolymer | 10.04% Carbopol ® Aqua SF2 | 25.22 | 13.55% |
| | 0.0194% NaOH | | |
| | QSP 100% deionized water | | |
| Base | 3.70% NaOH | 7 | 3.76% |
| | 19.77% Hepes | | |
| | QSP 100% deionized water | | |

The stability of the composition of Example 3 was studied at 50° C. for several days. The results in terms of pH and viscosity are provided in the following table:

| | Time (in days) | | | | |
|---|---|---|---|---|---|
| | 0 | 7 | 14 | 21 | 28 |
| pH | 7.36 | 7.11 | 7.24 | 7.04 | 7.02 |
| Viscosity | 1,180 | 1,236 | 1,492 | 1,188 | 1,196 |
| (mPa · s) | | | | | |

This composition proves to be advantageously stable for at least one month at 50° C. Indeed, the viscosity and the pH did not vary much over time and at 50° C. Consequently, the gel is not very acidified and retained its suspensive properties.

Furthermore, the composition according to Example 3 has an olfactory profile similar to that of a conventional perfuming composition, i.e. hydroalcoholic and comprising an identical perfume content.

### Example 4: Comparative Example

This example consisted in preparing two compositions which differ at the aqueous gel level suspending the droplets of perfume stabilized with a coacervate membrane.

The compositions of Example 4 consist of the following ingredients (the indicated percentages are expressed based on the total weight of the relevant phase):

| | | | Composition A | Composition B |
|---|---|---|---|---|
| | | | (Invention) | (Comparative) |
| Phase | Name | INCI name | % w/w | % w/w |
| Aqueous | Osmosed water | Aqua | qsp | qsp |
| phase | Carbopol 981 | Carbomer | 0.2% | 0.2% |
| (phase A) | Crosspolymer | Acrylates crosspolymer-4 | 2% | 0% |
| | Carbopol Aqua SF2 | | | |
| | Microcare ® PE | Phenoxyethanol | 0.8% | 0.8% |
| | Microcare ® emollient | Pentylene glycol | 2% | 2% |
| | PTG | | | |
| | Hepes | Hydroxyethylpiperazine | 1% | 1% |
| | | Ethane Sulfonic Acid | | |
| | | Hydro | | |
| | Edeta ® BD | Disodium EDTA | 0.01% | 0.01% |
| | NaOH | Sodium Hydroxide | 0.17% | 0.25% |
| Dispersed | Perfume | Perfume | 99.5% | 99.5% |
| phase | KF8004 | Amodimethicone | 0.5% | 0.5% |
| (phase B) | | | | |

| | | |
|---|---|---|
| Total | 100.00% | 100.00% |

The final compositions comprise 90% by weight of aqueous phase A and 10% by weight of dispersed phase B as described above, based on the total weight of said composition.

The compositions of Example 4 are prepared according to the following procedure:

Phase A:

Half of the water and the preservatives are mixed. Stirring is performed by means of a de-flocculator type blade for 15 min at 300 rpm.

The carbomer is incorporated with stirring and then this stirring is maintained for 1 h in order to ensure hydration of the latter.

For the composition A, the crosspolymer is added and stirring is performed until complete dispersion.

A solution is prepared separately with Hepes, the soda and the remainder of water which has just been added slowly to the preceding solution always with stirring.

Phase B:

In parallel, the phase B is prepared by mixing the amodimethicone and the perfume. This phase B is then incorporated to the aqueous phase A with moderate stirring.

Alternatively, the solution with Hepes, the soda and the remainder of water may be added after dispersion of phase B in phase A.

The stability of the compositions of Example 4 was studied at 50° C. for several days. The results in terms of pH and of viscosity are provided in the following tables:

| | |
|---|---|
| Time | Loss |
| (in days) | (absolute |

| | | 0 | 30 | loss) |
|---|---|---|---|---|
| pH | Composition A | 7.14 | 6.47 | −0.67 |
| | (invention) | | | |
| | Composition B | 7.17 | 5.94 | −1.23 |
| | (comparative) | | | |

| | |
|---|---|
| Time (in days) | Loss |

| | | 0 | 30 | (in %) |
|---|---|---|---|---|
| Viscosity | Composition | 2,172 | 2,149 | −1.06% |
| (mPa · s) | A (invention) | | | |
| | Composition B | 1,128 | 648 | −42.55% |
| | (comparative) | | | |

The viscosity is measured according to the method indicated above in the description.

As compared with the comparative composition B, the composition A according to the invention prove to be significantly more stable for at least one month at 50° C. Even if the pH of the composition A has decreased, the viscosity of the gel has remained stable over time and at 50° C.

This example therefore gives the possibility of showing that the use of a cross-linked copolymer or cross-linked polymer according to the invention advantageously gives the possibility of stabilizing compositions comprising a high amount of perfume.

Furthermore, the composition A according to Example 4 has an olfactory profile similar to that of a conventional perfuming composition, i.e. hydroalcoholic and comprising an identical perfume content.

1. A method for stabilizing at least one perfuming agent in a dispersion comprising at least 3% by weight of perfuming agent(s), the perfuming agent(s) being in the form of drops dispersed in an aqueous gel, said method comprising the addition of at least one cross-linked polymer or cross-linked copolymer into the dispersion, said polymer or copolymer comprising at least one unit derived from the polymerization of one of the monomers selected from the group consisting of acrylic acid, methacrylic acid, alkyl acrylate comprising from 1 to 30 carbon atoms and salts thereof,
said aqueous gel comprising water, a buffer having a pKa comprised from 4.0 to 9.0 and a base and further preferably at least one carbomer different from said cross-linked polymer or cross-linked copolymer, and
the size of the perfuming agent drops ranging from 0.2 μm to 5,000 μm.

2. A dispersion containing a dispersed phase comprising drops and a continuous aqueous phase as a gel, wherein:
the drops comprise at least one perfuming agent and a shell, said shell comprising at least one carbomer and/or a cross-linked copolymer acrylates/C10-30 alkyl acrylate;
the continuous aqueous phase comprises water, a buffer having a pKa comprised between 4.0 to 9.0, a base and at least one cross-linked polymer or cross-linked copolymer different from said carbomer and/or a cross-linked copolymer acrylates/C10-30 alkyl acrylate, said cross-linked polymer or cross-linked copolymer comprising at least one unit derived from the polymerization of one of the monomers selected from the group consisting of acrylic acid, methacrylic acid, alkyl acrylate comprising from 1 to 30 carbon atoms and salts thereof.

3. The dispersion according to claim 2, comprising from 0.1% to 10% by weight of cross-linked polymer(s) or cross-linked copolymer(s) based on the total weight of said dispersion.

4. The dispersion according to claim 2, wherein the cross-linked copolymer is selected from among the cross-linked copolymers of acrylic or methacrylic acid and of alkyl acrylates comprising from 1 to 4 carbon atoms.

5. The dispersion according to claim 2, comprising at least 3% by weight, of perfuming agent(s) based on the total weight of said dispersion.

6. The dispersion according to claim 2, wherein the drops further comprise at least one cationic polymer.

7. The dispersion according to claim 6, wherein the cationic polymer is a silicone polymer modified with a primary, secondary or tertiary amine function.

8. The dispersion according to claim 6, wherein the cationic polymer fits the following formula (I): 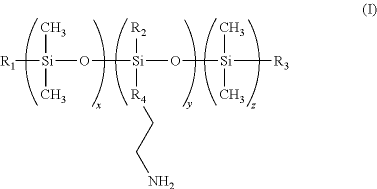
wherein:
R₁, R₂ and R₃, independent of each other, represent OH or CH₃;
R₄ represents a group —CH₂— or a group —X—NH— wherein X is a divalent C3 or C4 alkylene radical;
x is an integer comprised between 10 and 5,000;
y is an integer comprised between 2 and 1,000; and
z is an integer comprised between 0 and 10.

9. The dispersion according to claim 6, wherein each drop comprises from 0.01% to 10% by weight of cationic polymer(s), based on the total weight of the drop.

10. The dispersion according to claim 2, wherein the average diameter of the drops of the dispersed phase is comprised from 0.2 μm to 3,000 μm.

11. The dispersion according to claim 2, comprising from 0.05% to 5% by weight of carbomer(s) and/or cross-linked copolymer(s) acrylates/C10-30 alkyl acrylate based on the total weight of said dispersion.

12. The dispersion according to claim 2, wherein the buffer is selected from the group consisting of phosphate buffers, of 2-(N-morpholino)ethane sulfonic acid, of 2-amino-2-hydroxymethyl-1,3-propanediol, of 2-(bis(2-hydroxyethyl)amino)acetic acid, of 4-(2-hydroxyethyl)-1-piperazine ethane sulfonic acid, of sodium citrate and mixtures thereof.

13. The dispersion according to claim 2, comprising from 0.1% to 10% by weight of buffer(s), based on the total weight of said dispersion.

14. The dispersion according to claim 2, wherein the base is NaOH.

15. The cosmetic composition comprising a dispersion according to claim 2, associated with a physiologically acceptable medium.

16. A non-therapeutic method for cosmetic treatment of the skin comprising a step for applying on a keratinous material, of at least one layer of a dispersion according to claim 2.

17. A non-therapeutic method for cosmetic treatment of the skin comprising a step for applying on a keratinous material of a cosmetic composition according to claim 15.
